(19) United States
(12) Patent Application Publication
(10) Pub. No.: US 2021/0352905 A1
(43) Pub. Date: Nov. 18, 2021

(54) COMPOSITIONS AND METHODS TO DISINFECT, TREAT AND PREVENT MICROBIAL INFECTIONS

(71) Applicant: WIAB WATER INNOVATION AB, Malmo (SE)

(72) Inventors: Geir Hermod Almås, Snarøya (NO); Pål Rongved, Oslo (NO); Thomas Bjarnsholt, Oslo (NO)

(21) Appl. No.: 17/322,842

(22) Filed: May 17, 2021

Related U.S. Application Data
(60) Provisional application No. 63/048,815, filed on Jul. 7, 2020.
(60) Provisional application No. 63/025,743, filed on May 15, 2020.

(51) Int. Cl. A01N 59/08; A61J 1/20; A01N 37/02; A61K 31/19; A01N 25/04; A61K 33/14; A61K 9/06

(52) U.S. Cl. CPC A01N 59/08; A61J 1/2093; A01N 37/02; A61K 9/06; A01N 25/04; A61K 33/14; A61K 31/19

(57) ABSTRACT

The present invention provides new storable and stable disinfectant compositions comprising use of a solid precursor of an oxidized state of chlorine. The component in the device is devoid of stability issues, since the solid precursors are instantly to be dissolved in a pharmaceutically-acceptable diluent, adjuvant, or carrier, and combined with an activator (e.g. acetic acid or its salts), optionally combined with a viscosity enhancer, optionally combined with a dye. The main product is generated by breaking or opening seals, barriers or ports between an optional number of compartments in a multi-compartment device to mix the contents in the compartments, followed by gently squeezing or shaking the device to generate the disinfectant solution, wherein the resulting solutions can be taken out through a cap, port or opening on the multi-compartment device. The resulting compositions are useful disinfectants for treating a broad spectrum of pathogenic bacterial and/or viral, fungal or parasitic pathogens, denoted microbials.

### CROSS-REFERENCE TO RELATED APPLICATIONS

This application claims priority to, and the benefit of, U.S. Provisional Application No. 63/025,743, filed on May 15, 2020 and U.S. Provisional Application No. 63/048,815, filed on Jul. 7, 2020, the content of each of which is incorporated by reference herein in its entirety.

### FIELD OF THE INVENTION

The present invention relates generally to new compositions comprising combinations of a solid or liquid precursor of an oxidized state of chlorine and acetic acid or its salts, wherein such compositions are useful disinfectants for treating a broad spectrum of bacterial and/or viral, fungal and parasitic pathogens, collectively denoted microorganisms herein.

### BACKGROUND OF THE INVENTION

Microorganisms include bacteria, fungi, archaea, parasites, protozoa and viruses, and are among the earliest known life forms. These types of microorganisms may be either free-living or parasitic. Viruses can only be parasitic, since they always need other living organisms to reproduce.

Free-living microbes like bacteria or fungi have the potential to grow on surfaces or in host organisms, leading to infections in the host organism that may become pathological and possibly lead to death.

Infectious diseases are a leading cause of death worldwide and account for more than 13 million deaths annually including nearly two-thirds of all childhood mortality. Moreover, antibiotic resistance is increasing and is contributing to morbidity in a broad range of human diseases, pneumonia including tuberculosis or cholera. Of particular concern is the number of human pathogens developing multidrug resistance to conventional antibiotics. The introduction of new, more potent, derivatives of existing antibiotics provides only temporary solutions, since existing resistance mechanisms rapidly adapt to accommodate the new derivatives (Hoiby et al, Int. J. Antimicrob. Agents 2010). Although resistant Gram-positive bacteria pose a significant threat, the emergence of multidrug resistant (MDR) strains of common Gram-negative pathogens, such as Escherichia coli, are of particular concern. Pan-resistance or extreme drug resistance are now commonly used terms to describe clinically important isolates of Pseudomonas aeruginosa, Acinetobacter baumannii and Enterobacteriaceae that are resistant to virtually all antibiotics.

Viruses are also a significant concern in infectious epidemiology. Serious viral outbreaks, many of zoonotic origin, are becoming increasingly common. For example, the SARS (severe acute respiratory syndrome) and MERS (Middle East respiratory syndrome) outbreaks in the early-to-mid 2000s, the H1N1 pandemic in 2009, and the subsequent SARS CoV-2 pandemic in 2020 have focused attention on both treatment and prevention of the spread of these viral pathogens.

Many viruses that infect the respiratory tract are communicated via droplet infection. In that case, respiratory droplets containing virus are expelled by an infected person and picked up by others on direct contact or by contact with surfaces on which the droplets land. Typically, infection proceeds via the binding of the virus to receptors on mucosal or epithelial cells, followed by entry into the nose, eyes, ears, or mouth. In addition, other viruses are transmitted via aerosol particles containing the virus or are air borne. In either case, the virus may survive from hours to days after expression from an infected individual.

Infections can be caused by microorganisms selected from the group consisting of viruses, bacteria, fungi, spores, parasites and combinations thereof as described herein. The viruses can be exemplified by, but not limited to, one or more viruses, selected from the group consisting of adenoviruses, human immuno-deficiency virus (HIV), rhinoviruses, flu viruses (e.g., influenza A), hepatitis (e.g., hepatitis A). Further, the invention is effective against coronaviruses, responsible for Severe Acute Respiratory Syndrome (SARS) type viruses.

Examples of viruses are the SARS-CoV virus identified in 2002 as the cause of an outbreak of severe acute respiratory syndrome (SARS), the MERS-CoV virus, identified in 2012 as the cause of Middle East respiratory syndrome (MERS). The pandemic in 2020 is caused by SARS-CoV-2 virus, which is a novel coronavirus identified as the cause of coronavirus disease 2019 (COVID-19) that began in Wuhan, China in late 2019 and spread worldwide.

Further, problematic microbes are exemplified by, but not limited to, rotavirus, respiratory syncytial virus, herpes simplex virus, varicella zoster virus, rubella virus, and other common viruses. The bacteria can include, for example, one or more bacteria selected from the group consisting of Escherichia coli, Pseudomonas aeruginosa, Klebsiella pneumonia, Acinetobacter baumannii, E.coli, Staphylococcus aureus, Bacillus athrophaeus, Streptococcus pyogenes, Samonella choleraesuis, Shingella dysenteriae, Mycobaterium tuberculosis, and other known bacteria. Further, problematic fungi and yeasts can include, for example one or more of Candida albicans, Bacillus subtilis, Trichophyton mentagrophytes, and Bacillus athrophaeus.

Conventional compositions and methods for disinfection of inanimate surfaces or contaminated epithelia are not sufficient for the inactivation of all these infectious agents. Current forms of conventional disinfection compositions and methods may require long and impractical exposure times, or hazardous or corrosive solutions or vapors that cannot be used on expensive instruments or on living tissues, and thus fail to provide ready solutions to growing health risks from drug and disinfectant resistant agents.

It is clear that there is a significant unmet medical need for new therapeutics to treat resistant microbials and vira, particularly effective against key stages in the microbial entrance into mammalian cells via biological mechanisms, which at the same time are effective outside mammalian biology.

### SUMMARY OF THE INVENTION

The present invention provides new disinfectant compositions comprising use of solid precursors of oxidized states of chlorine, to be dissolved in a pharmaceutically acceptable diluents, adjuvants, or carriers, combined with activators, acting synergistically with the oxidized state of chlorine. The activator may preferably be acetic acid or its salts. Optionally, the resulting solution may be combined with a viscosity enhancer, and/or optionally combined with a dye. The different components according to the invention is contained separately in chambers in a multi-compartment device with 2-10 different compartments, and is mixed prior to use. The resulting solutions may be converted to viscous solutions or gels using viscosity enhancers. Such compositions are useful disinfectants for treating a broad spectrum of bacterial and/or viral pathogens.

### BRIEF DESCRIPTION OF THE DRAWINGS

FIG. 1 is a schematic illustration showing an exemplary multi-compartment or multi- chambered container for producing, storing, and dispensing a disinfectant composition according to embodiments of the present invention.

FIG. 2 shows the results obtained using sample solutions according to the present invention.

### DETAILED DESCRIPTION OF THE INVENTION

The present invention relates generally to compositions comprising a combination of a solid and liquid precursor of an oxidized state of chlorine and an activator, e.g. acetic acid or its salts, as well as one or more additional components. The use of such compositions acts as disinfectants for treatment of a broad spectrum of bacterial and/or viral pathogens on a variety of biotic and abiotic surfaces and environments.

Some preferred compositions of the invention may be in a solid form, multi-component (i.e., two-component, three-component, four-component, etc.) formulation that instantly generates the composition with long-term stability. This eliminates any issues related to shelf life seen with solutions of hypochlorous acid or chlorine dioxide described in the prior art. More specifically, the immediate generation of ready to use formulations of the oxidized chlorine species from solid precursors API-P may be performed in a multi-compartment device or container at the site of use. The multi-compartment device or container may be used for the preparation, dispensing, and long term, stable storage of prepared compositions consistent with the present invention. In particular, such multi-compartment containers described herein may have a number of compartments or chambers separately containing the components required to produce the compositions of the present invention. In example, the solid precursor of an oxidized state of chlorine and acetic acid or its salts, a viscosity enhancer, and a dye) and subsequently combine such components to prepare the composition at the desired time of use and on site.

By way of background, chlorine oxides, or oxidized chlorine (also referred to herein as “OC”), comprise a large class of chemical species, and are often found in nature, as well as biological systems in mammals. Chlorine oxides may also exist as neutral compounds or ions, so-called oxyanions. There are several oxyanions of chlorine, in which an oxyanion can assume oxidation states of +1, +3, +5, or +7 with the corresponding anions hypochlorite (ClO⁻), chlorite (ClO₂⁻), chlorate (ClO₃⁻), or perchlorate (ClO4⁻). The standard reduction potentials at a low pH of hypochlorous acid (HOCl) is +1.63, and for chlorous acid (HlO₂), the standard reduction potential is 1.64, while at basic pH, it is +0.89 and +0.78 respectively. At a pH of 5 to 7, reduction potentials are higher than +1.

Consequently, hypochlorite and chlorite are generally the most useful oxidation states with a potential to kill microbes and parasites. In particular, the chloride ion Cl⁻ is in the most stable oxidation state and is not reactive, nor is it effective as a disinfectant. Chlorate and perchlorate in oxidation states +5, and +7 are more reactive than the lower oxidation states, and may be more difficult to handle.

The hypochlorite ion has the chemical formula ClO⁻, where chlorine (Cl) is in oxidation state +1, which is a potentially unstable oxidation state since the low-energetic oxidation state of Cl is −1. Both the hypochlorite ion and the chlorite ion combine with a number of cations to form hypochlorites and chlorites, as the salts of these oxidized chlorines. Common examples include sodium hypochlorite (household bleach) and calcium hypochlorite, the main active ingredient of commercial products including bleaching powder, chlorine powder, or chlorinated lime, generally used for water treatment (e.g., swimming pools and the like). The chlorite and hypochlorite ions also referred to herein as the “main chlorine oxides”, are useful in various contexts. Sodium chlorite and hypochlorite are strong oxidizing agents, and have been used in water purification, disinfection, as well as bleaching and deodorizing animal products.

Because of sodium hypochlorite producing highly toxic chlorine gas under acidic conditions, commercially available aqueous solutions for household purposes are strongly basic solutions, with the pH adjusted using sodium hydroxide.

Chlorite and hypochlorite are also useful for treatment of various diseases or conditions. For example, chlorite and hypochlorite are useful in treatment of infections, as described in U.S. Pat. Nos. 4,725,437 and 4,851,222. Chlorite and hypochlorite may also been used to treat HIV, recurrent prostate cancer, cystitis, and chronic active hepatitis C disease (see, for example, McGrath et al., Development of WFlO, a novel macrophage-regulating agent, Curr. Opin. Investig. Drugs, 3(3):365-73 (March 2002)). Chlorite and hypochlorite have also been described for use in treating oral or periodontal diseases or conditions, such as inflammation of the gingiva (see, for example, U.S. Pat. No. 6,350,438).

Hypochlorous acid (HOCl) is widely used as broad-spectrum household and industrial disinfectant and deodorizer. Hypochlorous acid is a weak acid that is known to rapidly inactivate bacteria, algae, fungus, and other organics, making it an effective agent across a broad range of microorganisms. Additionally, since hypochlorous acid is a weak acid and since people naturally produce certain compounds that allow them to tolerate hypochlorous acid, it is generally not harmful to people. Due to the combination of its biocide properties and its safety profile, hypochlorous acid has been found to have many beneficial uses across many different industries, such as the medical, foodservice, food retail, agricultural, wound care, laboratory, hospitality, dental, or floral industries.

Hypochlorous acid is formed when chlorine dissolves in water. In particular, the acidification of hypochlorite generates hypochlorous acid, where the chlorine atom is in oxidation state +1. Hypochlorous acid exists in equilibrium with chlorine gas, which can escape from solution. The equilibrium is pH-dependent, as illustrated in the following equation (Equation 1):

Cl₂+H₂O [symbol] HOCl+Cl⁻+H⁺ [symbol] ClO⁻+Cl⁻+2H⁺ (1)
Increasing pH→

With reference to the above equation (Equation 1), a high pH drives the reaction to the right, promoting the disproportionation of chlorine into chloride and hypochlorite, whereas a low pH drives the reaction to the left, promoting the release of chlorine gas (Cl₂).

Chlorine gas (Cl₂) is a toxic and powerful oxidizing agent. One inconvenient property of chlorine gas is the tendency to insert Cl into hydrocarbons, to form mono- or poly-halo-alkanes and other chlorinated hydrocarbons in radical reactions (See Barhorst and Kubiak, Environ. Sci. Pollut. Res. (2009) 16, pp 582-589). Halogenated hydrocarbons are toxic environmental pollutants. This is a potential hazard, since halogenated hydrocarbons are pollutants and potentially considered carcinogenic. Thus, chlorine gas is not a desired compound to use at higher concentrations in nature or in medical applications. At lower concentrations, chlorine is contributing in the equilibrium to the powerful disinfectant properties of hypochlorous acid with low reasonable risk of medical side effects. However, as long as pH in solutions of hypochlorous acid is controlled and kept in the interval of 4-8, the amount of Cl₂ is negligible.

The antimicrobial effect of hypochlorous acid is highly pH dependent, and provides the highest level of activity at a pH value of 5 to 5.5, which is about 2 pH units away from physiologic pH (7.1-7.5). This means that a hypochlorite solution should ideally be buffered with a weak organic acid and its corresponding metal salt, to have its maximum antimicrobial activity.

In general, formulations containing chlorine oxides are effective antimicrobial agents, with proven antimicrobial and antiprotozoal properties that are useful in disinfectant technologies concerning human and animal health. However, formulations in the prior art have drawbacks. For example, the weak acid HOCl is unstable and impure when produced under conventional conditions. Consequently, there is a need for a more controlled, and immediate preparation processes that can furnish chlorine oxides on site with a stability that permits the intended short-term use.

While the alkalinity of household hypochlorite bleach may be around a pH value of 12, solutions of HOCl are weakly acidic with a pKa value of about 7. Formulations of HOCl in a pH interval around physiological pH 7 are therefore more compatible with medical applications for which bleach is damaging and hazardous to users and to surfaces to which it is applied. Thus, it is of utmost importance to keep pH in the range 4 to 9 to avoid damage to biological tissue or vulnerable surfaces.

In a therapeutic technology, an active pharmaceutical ingredient (also referred to herein as “API”), is understood to be the chemical component or components generating the respective therapeutic effect. A weakness of the formulations in prior art, where hypochlorous acid is the only active API, is the absence of a biocompatible activator and pH-stabilizer that simultaneously acts both as a buffer and as an antimicrobial agent, preferably also in synergy with the API.

Another weakness of prior art technologies is that many of the water solutions in these cases has been produced by electrolysis of isotonic 0.9% NaCl/H₂O (normal saline) and has no buffering capacity because a mixture of a weak biocompatible organic acid and its salt is not present. Further, electrolysis is a rather complicated and sometimes inconvenient method for preparation of the antimicrobial solutions (i.e., in war zones, tourist resorts, or areas of catastrophes or pandemics).

A further weakness of prior art chlorine oxide-based formulations is a lack of stability, based, at least in part, on the fact that chlorine oxides gradually deteriorate and decompose within the first few months of storage, unless stored cold and in the absence of light and oxygen. Thus, pharmaceutical shelf-life stability of solutions of chlorine oxides cannot be achieved at ambient conditions.

Yet another weakness of the chlorine oxide-based formulations in the prior art is the lack of a controlled ionic strength that is adapted to the use of a given formulation. The administration of any given formulation including a chlorine oxide to a mammal ideally requires an ionic strength of 300 mOsm to be iso-osmolal to body fluids, equivalent to about a 150 mM concentration of NaCl.

Hypochlorous acid may be an attractive compound to include in antimicrobial solutions, as neutrophils in the body produce it naturally in pure form in vivo. Further, circulating monocytes, tissue-resident macrophages, and microglial cells in mammals also produce hypochlorous acid to inactivate pathogens within phagocytic vesicles and in the extracellular space around phagocytes in tissues. Thus, hypochlorous acid is a natural biological compound produced by mammals in relatively high concentrations. Activated neutrophils can mobilize their primary granule enzymes to the cell surface. For example, as described by Hirche et al, im J. Immunol. (2005); 174:1557-1565, activated neutrophils use myeloperoxidase (MPO) uses H₂O₂ catalyzed by NADPH oxidase to generate HOCl.

Considering the large number of neutrophils that accumulate in inflamed tissues such as lungs of patients with acute respiratory distress syndrome, the concentrations of HOCl that can be of clinical use are of notable physiologic relevance. It is well known in prior art that 5×10⁶ activated neutrophils can generate as much as 100 μM HOCl within 2 hours. Thus, the mammalian biological system may handle concentrations of at least 100 μM HOCl without pathologic consequences.

Another chlorine oxide useful as an API in antimicrobial formulations is chlorine dioxide, wherein the chlorine atom is in oxidation state +3. The main reaction of sodium chlorite is the generation of chlorine dioxide, as illustrated in the following equation (Equation 2):

5NaClO₂+4HOR [symbol] 5NaOR+4ClO₂+2H₂O (2)

Referring to the above equation (Equation 2), HOR is usually a mineral acid, such as HCl or citric acid, since a source of protons is needed to convert sodium chlorite, first to chlorous acid, and then to chlorine dioxide, which is a highly water-soluble gas at room temperature.

An advantage of chlorine dioxide is that it cannot generate chlorine gas, Cl₂ which is known to react to chlorinated hydrocarbons, e.g. trihalo-methanes, which are toxic environmental pollutants. Another advantage of chlorine dioxide is that the activity as a disinfection agent or stability of its water solutions is not pH-dependent.

Chlorine dioxide is generated from sodium chlorite, is approved by FDA under some conditions for disinfecting water, and is used to wash fruits, vegetables, and poultry. Sodium chlorite, NaClO₂ is a solid precursor of chlorine dioxide, and is sometimes used in combination with zinc chloride. It also finds application as a component in therapeutic rinses, mouthwashes, toothpastes and gels, mouth sprays, as preservative in eye drops, and in contact lens cleaning solution under the trade name Purite.

Chlorine dioxide is also used for bleaching and stripping of textiles, pulp, and paper. It is also used for disinfection of municipal water treatment plants after conversion to chlorine dioxide. Chlorine dioxide is used for sanitation of the hard surfaces, which come in contact with food and as a wash or rinse for a variety of foods including red meat, poultry, seafood, fruits and vegetables. Because the chlorine oxide compounds are unstable even when properly prepared, there is no measurable residue on food after disinfection. Chlorine dioxide also is used as a teat dip for control of mastitis in dairy cattle.

The U.S. Army Natick Soldier Research, Development and Engineering Center produced a portable “no power required” method of generating chlorine dioxide, known as ClO₂ gas, an efficient biocide available for combating contaminants, which range from benign microbes and food pathogens to Category A bioterror agents. In the weeks after the 9/11 attacks when anthrax was sent in letters to public officials, hazardous materials teams used ClO₂ to decontaminate the Hart Senate Office Building, and the Brentwood Postal Facility.

In addressing the COVID-19 pandemic, the U.S. Environmental Protection Agency has included ClO₂ as an agent that met its criteria for use in environmental measures against the causative corona viruses (see (US EPA, OCSPP (Mar. 13, 2020) “List N: Disinfectants for Use against SARS-CoV-2”. US EPA Retrieved Mar. 28, 2020, titled “How we know disinfectants should kill the COVID-19 coronavirus”, in Chemical & Engineering News, Retrieved Mar. 31, 2020).

However, such formulations in these cases are also subject to the stability issues described above, since these technologies also are devoid of a biocompatible activator and stabilizer of a precursor of the chlorine oxide that at the same time acts synergistically in the antimicrobial action of the final formulation.

In summary, the main challenge in prior art of the medical use of solutions of chlorine, in higher oxidation states than −1, is stability, or lack thereof, since these chemical species are in a higher energy state and tend to return to the chloride ion Cl⁻ and will decompose in solution at ambient temperature. This prohibits the required shelf life stability at ambient conditions of pharmaceutical formulations and medical devices of chlorine oxides. Accordingly, a proper shelf life, as required for medical devices and drugs, is difficult to achieve for solutions of chlorine oxides. This inherent limitation to all oxides of chlorine restricts transport and storage, especially at higher temperature in areas with variable temperature, light humidity, and atmospheric gases.

The present invention recognizes all these drawbacks associated with prior art compositions using chlorine oxides. In particular, the present invention provides compositions comprising a combination of solid precursors of oxidized states of chlorine (OC) and activators providing a source of protons. A preferred example of an activator is acetic acid or its salts, wherein the disinfectant compositions of the invention are instantly formed in a controlled and immediate process at the site of use with a stability that permits the intended short-term use. Such compositions are useful disinfectants for treatment of a broad spectrum of microorganisms. In particular, when the active pharmaceutical ingredient is generated from stable, solid precursors, referred to hereinafter as “API-P” of chlorine oxides at the site of use, the inclusion of e.g. acetic acid as an activator that simultaneously is buffering the solution or gel to a biocompatible pH value, the stability issue in prior art is no longer present.

As previously described, the technical solutions in the prior art fail to address how to secure an ionic strength or osmolality of the final antimicrobial solution biocompatible with biological fluids. Even further, the prior art fails to how to regulate and increase contact time and persistence of the API in region of therapeutic interest, e.g. by regulating rheology and fluidity. Yet still, the prior art fails to provide a relatively simple, yet effective, means of monitoring an oxidation state of the API and visual indication of where the API has been applied during mixing of a disinfectant composition.

Additionally, in some embodiments, compositions of the present invention may further include the use of a viscosity enhancer (also referred to herein as “VE”) and/or include a combination of a solid precursor of an oxidized state of chlorine and the activator, e.g. acetic acid or its salts.

Another embodiment of the invention is the inclusion of a dye in the formulation, preferably e.g., a redox sensitive dye, with a colour that varies with the oxidation state of the chlorine atom, the advantages of which address the drawbacks described with respect to prior art compositions.

In particular, some preferred compositions of the invention may be in a solid form, multi-component (i.e., two-component, three-component, four-component, etc.) formulation, separated by breakable walls or barriers that instantly generates the composition with long- term stability. This eliminates any issues related to shelf life seen with solutions of hypochlorous acid or chlorine dioxide described in the prior art.

More specifically, the immediate generation of ready to use formulations of the oxidized chlorine species from solid precursors API-P may be performed in a multi-compartment device or container at the site of use. The multi-compartment device or container may be used for the preparation, dispensing, and long term, stable storage of prepared compositions consistent with the present invention. In particular, such multi-compartment containers described herein may have a number of compartments or chambers separately containing the components required to produce the compositions of the present invention. In example, the solid precursor of an oxidized state of chlorine and the activator, e.g. acetic acid or its salts, a viscosity enhancer, and a dye is mixed, and subsequently the composition generates at the desired formulation of the disinfectant at the desired time and site of use.

Acetic acid is an abundant natural compounds found in nature and in biological systems of mammals in different tissues. It is a by-product from bacterial fermentation of carbohydrates in the food, and I nature. With a pKa of 4.7, it exists in the form of its alkali metal salt in a biological system.

Sodium acetate is non-toxic and is allowed in drug formulations for oral and parental use. The bactericidal effect of acetic acid is well known. It has a documented effect against problematic Gram-negative bacteria such as P. vulgaris, P. aeruginosa and A. Baumannii and others, as described by Ryssel et al in Burns (2009), 35, pp 695-700. The microbiological spectrum of acetic acid is wide, even when tested at a low concentrations of 0.5-3%. The concentrations of acetic acid that eradicated a pre-formed biofilm ranged from 0.10% to 2.5%, as described by Halstead et al in PLOS ONE (2015), pp 1-15.

Thus, acetic acid and its metal salt are very attractive compounds to use in antimicrobial formulations because of its ability to act as a buffer together with its metal salt for stabilization of pH.

Further, in addition to its antimicrobial properties, acetic acid is attractive because it cannot be oxidized further by oxidizing agents, such as an OC, and because of its endogenic nature in high concentrations biochemically speaking.

Accordingly, the multi-compartment container enables practical use in mixing the components necessary to generate the active solution of the API instantly and at the site of use. It should be noted that, to secure an ionic strength or osmolality of the final antimicrobial solution to adapt to the osmolality on the region of use in the case of medical applications, a pre-calculated amount of NaCl can be included in the multi-compartment device, dependent on the planned use.

A preferred embodiment of the invention is inhalation of selected solutions according to the invention for fighting virus infections un the respiratory systems of mammals. Thus, any nebulizers or inhalators, generally used for the treatment of cystic fibrosis, asthma, COPD and other respiratory diseases or disorders, converting liquids into aerosols are useful in the present invention. The devices are often employing compressed air or ultrasonic energy to generate atomization of the disinfectant solutions. pressurized metered dose inhalers (pMDIs), dry powder inhalers (DPIs), slow mist inhalers (SMIs) of any kind, are Especially useful, e.g. as described by Prajapati et al. in IJPSR, 2019; Vol. 10(8): 3575-3582. Any electrostatic or non-electrostatic inhalators, e.g. the VORTEX or Pari or Sympotec are also useful to practice the invention.

The pre-loaded multi-compartment container is devoid of stability issues, produces a highly broad-spectrum antimicrobial solution upon mixing of the components, and leaves only biocompatible inactive chemical species already found in human biology or in nature.

As noted above, the activation of the API is effected using an activator, e.g. acetic acid, which acts synergistically with oxidized chlorine against microbes, and further maintains acidity in pH range between 4 and 8. This novel method and the formulations thereof avoids the inherent lack of long-term stability of oxidized chlorine OC in solution, since there is no need to store the disinfectant composition as a water solution.

Another advantage of the present invention is the option to add other compounds that will aid in application. For example, in wound healing applications, there is a need to increase the viscosity (μ) of the product on the skin to prolong contact time. This is solved by the use of a water-soluble or dissolvable viscosity enhancer (VE) that chemically cannot be oxidized by the API, thereby providing improved regulation of contact time and persistence of the API in a region of therapeutic interest. The VE ensures that the rheology and fluidity is adapted to the respective method and region of disinfection, to generate a solution with full fluidity or a gel. The VE may include, for example, a water-soluble gelling agent such as poly acrylic acid, polyethylene glycol or any other oligomer or polymer that cannot be oxidized by the API.

Additionally, the composition may include a one or more dyes, identified in a group of reduction-oxidation dyes (also referred to herein as “ROD” or “RODs”), wherein the color and intensity is dependent on the oxidation state of the oxidized chlorine. It should be noted that, in addition to providing a visual indication (i.e., by way of color) of the oxidation state of the chlorine atom, the RODs further provide an antimicrobial effect of their own. This enhances the synergistic action between the components in the formulation in a novel way. The ROD is able to maintain its color for a period of time sufficient to monitor the oxidative activity of the API, oxidized chlorine, and further provide a visual indication of the region wherein the formulation has been applied, thereby addressing the drawbacks of prior art.

Advantages of the invention, as well as additional inventive features, will be apparent from the description of the invention provided hereinafter.

The oxidized chlorine species denoted OC, having the general formula denoted below:

Mⁿ⁺[Cl(O)ₓ]ₙⁿ⁻

wherein M can be any alkali metal, alkaline earth metal or transition metal ion, n is an integer 1-5, x is an integer 1-4, y is an integer 1-2.

If M=Na, n=1, x=1, the API-P is the solid NaOCl. If M=Ca, n=2, x=1, the API-P is the solid Ca(OCl)₂. If M=Na, n=1, x=2, the API-P is the solid NaClO₂. If M=Ca, n=2, x=2, the API-P is the solid Ca(ClO₂)₂. In the case x=3 or 4, the API-P generates the more reactive chlorate and perchlorate species.

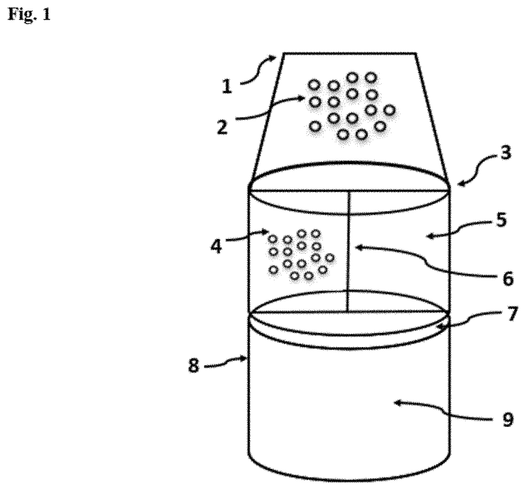

One non-limiting example is instant generation of hypochlorous acid from sodium-, or calcium-hypochlorite in the cap 2 according to FIG. 1, with a solution of sodium acetate buffer in compartment 4 providing a ready to use solution of the API hypochlorous acid with a pH between 5 and 6 in compartment 9, optionally with a color and a viscosity enhancer.

Another non-limiting example is calcium di-hypochlorite Ca(OCl)₂, which is a stable and water-soluble API-P for HOCl, which is being produced and sold in ton-scale as a pool disinfectant. It is instantly soluble in water, and only leaves calcium hydroxide, which present in nature and biology, is used in food as E-number E526, and I generates HOCl, one of the two the active ingredient in the present invention, which degrades to and biocompatible species containing hydrogen and oxygen.

Another preferred embodiment of the present invention is a solid precursor of oxidized chlorine is tetrachloro-decaoxide (TCDO), CAS no. 92047-76-2, known as WF10 or stabilized solutions of OXO-K993, prepared as described by Meuer et al in CA2616008. It can be prepared by combining alkaline or alkaline earth salts of the chlorite ion ClO₂⁻ with excess oxygen in water.

Thus, one advantage of the present invention is that the solid form precursors API-P in a dry and water-free quality is devoid of pharmaceutical stability issues, thus the present invention solves one of the main technical problems in prior art.

A main aspect of the invention is the combination of the API-P with a molecule comprising a carboxylic acid functionality —COOH, a sulfonic acid functionality —SO₃H, a phosphoric acid functionality —PO₃H or a boric acid functionality —B(OH)₂, hereinafter defined as the activator of the API-P in the formulation. The activator has the general formula R₁XOₙ(R₂)ₘ wherein the group Ri may be a group comprising 1-10 hydrogenated carbon atoms, optionally substituted with amino, amido, carboxylic or hydroxy groups. The group X may be a carbon, phosphorous or sulfur atom, n and m may be an integer 2 or 3 and R₂ may be a proton H, or any alkali metal, alkaline earth metal or transition metal ion. The nature of the substituents in the formula varies according to use and chlorine species, and may be any compound comprising an amino group, e.g. ammonia, an amino acid, e.g. taurine or a therapeutic drug increasing the synergistic potential of the formulation. The activator may be any combination or mixture of two or more compounds as defined by the general formula R₁XOₙR₂.

Preferred non-limiting examples are carboxylic acids R₃COOH, wherein R₃ is H, or a linear or branched saturated or unsaturated hydrocarbon chain with 1-24 carbon atoms, optionally substituted with hydroxyl groups. Non-limiting examples of activator may be acetic acid, citric acid, tartaric acid, lactic acid, hippuric acid, maleic acid, boric acid, sulfuric acid, phosphoric acid, boric acid, 3-(N-morpholino)propanesulfonic acid (MOPS). 2-(carbarnoylmethylamino)ethanesulfonic acid (ACES), 2-(carbamoylmethylamino)ethanesulfonic acid (ADA), 2-(carbamoylmethylamino)ethanesulfonic acid (bicine), piperazine-N,N′-bis(2-ethanesulfonic acid, PIPES), or any amino acid.

Taurine is especially preferred, since it is the endogenic amino acid normally moderating the effect of OC in the body, and may be combined with OC to form endogenous N-chloro-amino acids like ClNH—CH₂CH₂—SO₃H, which in itself has antibacterial properties.

Acetic acid is even more preferred since it is also an endogenic substance, has antibacterial properties, has very low toxicity and forms buffers in admixture with is metal salts, and is used as a non-limiting example in the further description of the invention.

An advantage of the invention is that the solid multi-component products according to the invention is not hampered with stability issues in a pharmaceutical or medical device setting, regardless of temperature, air, humidity, light, oxygen or other ambient conditions, since the API-Ps are solid and commercially available in large scale.

A further embodiment of the invention is that the API-Ps are instantly soluble in water, and instantly reaches physiologically acceptable pH and ionic strength in the final solution in combination with acetic acid and/or its salts. The products after disinfection are all endogenic species already present in the human biology or in nature.

Another embodiment is that instant generation of the active disinfection agent according to the invention on site of use will have significant and great influence on the versatility of product applications. It opens for a much more flexible product packaging technology, since the size and amount of substance in the starting package is freely variable, dependent on use.

Small, stable one-dose single-use two or three-component devices will be available, ideally suitable for tourists, catastrophic zones, with military personnel or microbial pandemics. Further, design of large flasks or tanks comprising the API-P is useful in agricultural settings, aquaculture industry or military operations, suitable to disinfect larger areas.

Viscosity Enhancers for Preparations o Viscous Solutions and Gels

A preferred embodiment of present invention is the option to include other compounds in the device or container that will aid user-friendliness of the product. For wound healing or skin disinfection applications, there is a need to increase the viscosity (μ) of the product on the skin to prolong contact time. The viscosity enhancer, VE, solves this medical need.

Preferred groups of VE according to the invention are water-soluble gelling agents that the API is not oxidizing since all oxygen-containing functional groups in the VE are in their highest oxidation state. The gelling agents provide prolonged persistence of the API at the area of interest, e.g. mammalian skin.

Examples of gelling agents according to the invention include, but are not limited to, poly acrylic acid (Carbomer), polyethylene glycol or any other oligomer, polymer or block-copolymer thereof. Further, the viscosity enhancer may be selected from, poly(acrylic acid)-acrylamidoalkylpropane sulfonic acid co-polymers, phosphino polycarboxylic acids and poly(acrylic acid)-acrylamidoalkylpropane or sulfonic acid-sulfonated styrene terpolymers.

Thus, a preferred aspect of the invention is the use of polymers, such as any type of acrylate copolymer, which are well known to those skilled in the art, can function well in the formulation of the invention in concentration ranges 0.01-5%. Acrylate copolymers are homo- and co-polymers of acrylic acid cross-linked with a polyalkenyl polyether. Acrylate copolymers come with a variety in graft density. They vary in terms of their ability to oxidize and how many grafted chains there are per polymer. One possible cross-linker is pentaerytritol, which is very stable, and so it is a good choice for use with the present invention. Polyacrylic acid (PAA) polymers that are known to stabilize formulations of H₂O₂, can be used with the present invention. (see Schmucker-Castner & Desai, 1999, “Rheology Modification of Hydrogen Peroxide Based Applications Using A Cross-linked PAA polymer,” Int J Cosmet Sci 21(5):313-25).

The polymer-stabilized solutions of OC according to the invention have applications in many contexts, e.g. in wound treatment, aseptic packaging, electronics manufacture, and pulp and paper bleaching. The formulation of the API is compatible with formulations as gels or viscous fluids, which may be applied to target surfaces, either inanimate or representative of the infected epithelial mucosal or skin surfaces of infected persons or animals, so as to ensure prolonged and intimate contact with the necessary levels of API. Non-viscous formulations of the API may also be dispersed into the air in confined spaces as a mist in order to achieve environmental disinfection, or for inhalation purposes for treatment of respiratory diseases.

In example, a concentration of the poly acrylic acid Carbomere has increasing viscosity in the concentration 0.01-0.1%. If desired, it forms regular gels in the concentration range 0.1-1%. To dissolve the VE according to the solution, a mixer, stirrer or sonicator must be used to dissolve it in the disinfective formulation. The degree of gelling can be even more increased using a biocompatible base, e.g. the pharmaceutical buffer triethanolamine, or other biocompatible amino alcohols.

Antibacterial Redox-Sensitive Antibacterial Dyes as Indicators

A further innovative and, in the context of disinfection technologies, novel aspect of the present invention is the use of reduction-oxidation dyes, for clarity hereinafter denoted ROD, wherein the color and intensity is dependent on the oxidation state of the OC. Even more advantageous, the identified RODs have antimicrobial effect in their selves, increasing the antimicrobial synergy between constituents according to the invention. If the standard half-cell potential of the ROD has a lower positive value than OC, the color of the formulation will be maintained as long as the OC is active. Thereby, the color visualizes the region wherein the formulation has been applied and where there is active OC. This is especially advantageous when e.g. the formulation according to the invention is used in treatment of mastitis, where large packs of cattle needs to be treated for mastitis; the colored formulation according to the invention visualized which animals have been treated. Further, employment of the opposite type of indicator, where the color appears when the oxidizing power of the OC is vanishing, is also useful

Non-limiting examples of suitable dyes useful in the invention, are pH-independent dyes, visible in the presence of an OC. Preferred examples are N-phenylanthranilic acid (violet-red), N-ethoxychrysoidine (cyan), o-dianisidine (red), sodium diphenylamine sulfonate (red-violet), diphenylbenzidine (violet), diphenylamine (violet) and viologen, which is colorless in the presence of an OC, but deep blue in the absence of an OC.

Examples of pH-dependent dyes that are deep blue in the presence of an active OC, but colorless in the absence of the OD are sodium 2,6-Dibromophenol-indophenol or Sodium 2,6-Dichlorophenol-indophenol, sodium o-cresol indophenol, thionine (syn. Lauth's violet), methylene blue, Gentian Violet, indigotetrasulfonic acid, indigo carmine (syn. Indigo-disulfonic acid), indigomono sulfonic acid. Examples of dyes that are red or red-violet in the presence of an OC are phenosafranine, Safranin T, neutral red and dialkyl-p-phenylenediamine (SPD, red violet).

Many of these dyes have antibacterial effects in their selves, i.e. methylene blue (MB) and Gentian Violet (GV), and combinations of them have been used as antibacterial dyes in foams in wound dressings in combination with polymers like polyvinyl alcohol or polyurethane, e.g. as described by Edwards in Advances in Wound Care (2016), 5, pp 11-19.

A particularly useful class of dyes useful in the present invention is microbial phenazines, which are pigmented, redox-active, nitrogenous aromatic compounds with metabolic, ecological and evolutionary significance, see e.g. Chincholkar, S. & Thomashow, L. Microbial Phenazines: Biosynthesis, Agriculture and Health (eds Chincholkar, S. & Thomashow, L.) 1-243 (Springer, 2014). All these distinctive features make them attractive microbial metabolites, useful as dyes in drug formulations. Reports of over 100 natural and 6000 synthetic phenazines to date exhibiting promising bioactivities including antimicrobial, anticancer, anti-parasitic, and insecticidal and biocontrol properties are available.

An even more attractive class of phenazines include the bis-N-oxide phenazines, with even stronger antimicrobial properties than their parent phenazines. Most of these compounds are natural compounds produced by bacteria, and are hetero-aromatic N-oxidized compounds, hereinafter denoted HANOX. In addition to being redox dyes, RODS, the HANOX compounds are useful in the present invention because their color is dependent on the oxidation state of the OC. At the same time, HANOX in prior art show to possess broad-spectrum anti-microbial activity, e.g. as described by Leimgruber et al in U.S. Pat. No. 3,822,265.

Further, the phenazine derivatives provided by U.S. Pat. No. 3,822,265 have a broad-spectrum fungicide activity. In particular, they demonstrated a high activity against a wide variety of bacterial, yeasts and fungi such as Streptococcus agalactiae, Staphylococcus aureus, Escherichia coli, Corynebacterium pyogenes, Moraxella bovis, Pseudomonas aeruginosa, Candida albicans and Microsporum canis. Thus, the phenazine derivatives are particularly useful in the treatment of animal diseases of microbial origin in agriculture.

A surprising finding with these derivatives is their lack of injurious effects to the body tissue under the conditions of use, making them particularly suitable for topical application, preferably employed in amount ranging from 0.05 per cent to 1.0 per cent by weight of the composition.

They are of particular value in topical applications, e.g. in solid or gel formulations including finely divided powders and granular materials and in liquid formulations including solutions, suspensions, concentrations, tinctures, slurries and aerosols, creams, gels, jellies, ointments and pastes.

An even more preferred group of HANOX, useful in the present invention, is described by Viktorsson et al in WO2015063516A2 and WO2018109504A1. This technology describes RODs possessing red-ox properties and broad-spectrum antimicrobial properties. As described by Viktorsson et al in Bioorg. Med. Chem. (2017)25, pp 2285-2293, this specific class of HANOX is especially attractive since they show lower toxicity in mammals compared to the compounds described in U.S. Pat. No. 3,822,265, and may be used with low risk of toxic reactions in the hos of the infection.

Methylene blue is another particularly preferred dye useful in the invention, since FDA has approved it as an excipient in drug formulations and it has antibacterial properties and its effects as a therapeutic agents can be enhanced using photodynamic therapy.

Multi-Compartment Devices Useful in the Invention

FIG. 1 is a schematic illustration showing a schematic, non-limiting and exemplary multi-compartment device for instant generation of a disinfectant solution of the API with excipients in a water solution according to the embodiments and methods of the present invention.

As illustrated, the multi-compartment device 8 schematically illustrates as a non-limiting example. The design of the device, including the number of compartments, can be adapted to the actual use. The device 8 consists of a screw cap 1 with the primary compartment, containing the solid precursor of the API, denoted API-P in a dry form (2). The screw cap I has the ability to open the seal or port 3 by turning the cap in one direction, letting the API-P into the second compartment 4, comprising a water solution of the activator also comprising a pre-calculated amount of sodium chloride to render the final osmolality of the solution to be iso-osmolal with body fluids. To gain the desired final pH, the activator and optionally a pre-calculated amount of its metal or amino acid salt in water may optionally be pre-loaded into compartment 4, 5 or 10. The smaller grains in 4 illustrate that the API-P is rapidly dissolving in the activator solution to generate the API. The third compartment 5 is optional, to contain a solution of a redox dye (ROD), dependent of the technical use of the respective device. Compartment 4 and 5 are separated by a wall 6. Compartment 4 and 5 are also separated from compartment 10 by a breakable septum or wall 7. Optionally, a fourth compartment at the same level as 4 and 5 can contain an amino acid, e.g. an essential or non- essential amino acid or taurine for stabilization of the API. For simplicity, in the present illustration, the fourth compartment 10 may optionally be pure water, the activator solution. When eventually turning the screw cap 2 in the opposite direction, the ready-to-use disinfectant solution is released from the device and may be applied at the region of interest for disinfection. A main aspect of the invention is the solid precursor API-P in the screw cap 2, which is the oxidized chlorine species. The resulting solution from the multi-compartment device may eventually be used to produce a solution of the viscosity enhancer (VE), in a water solution.

Multiple multi-chamber and multi-compartment devices are in use in different medical, domestic and agricultural applications. Many of these are useful in the present invention, and may be a dual- or multi-chamber bottles, bags, syringes, inhalators, hand disinfection devices, spay bottles or flasks, or tanks, constructed of hard or soft materials, e.g. plastic, rubber, water-proof paper or metal. The therapeutic formulation in can be easily activated bedside from the device, in the field or in domestic setting, without complicated mixing procedures, and can be stored at ambient temperatures. It can be combined with automated dispensing systems, easily labelled, automatically charted via barcoding.

The multi-compartment device according to the invention is a closed system and may be designed to eliminate mixing errors, to avoid undesired exposure to patients and personnel, and meets the Joint Commission and USO 797 guidelines.

Non-limiting examples of design useful in the invention are the Duplex Container from B Braun, the Credence Companion Safety Syringe System, the Dual-Mix multi-chamber bags or the Easyrec kit comprising a screw cap releasing a solid or mixture of solids for mixing into one or more fluid phases to generate the ready to use formulation of the API.

Use of the Invention for Antimicrobial Purposes with Photodynamic Therapy

Bacterial elimination using antimicrobial photodynamic therapy (aPDT) has been shown using the alternative therapeutic modality in peri-implantitis treatment. In Photodiagnosis and Photodynamic Therapy (2019), 25, pp 7-16, Huang et al described dose- dependent and pH-dependent bactericidal effects of methylene blue (MB)-mediated aPDT at eliminating Gram-negative (P. gingivalis and A. actinomycetemcomitans) and Gram-positive (S. mutans) bacteria on sandblasting, large-grit and acid-etching (SLA)-pretreated titanium alloy. However, the test formulations did not comprise an OC to enhance the therapeutic effect even further.

Thus, another preferred embodiment of the present innovative formulation comprising an OC, acetic acid or its salt, optionally a viscosity enhancer, is the inclusion of a ROD exemplified by methylene blue for the use of photodynamic therapy, e.g. to improve wound healing or bacterial infections in mammals. In this case, the site of administration of the product according to the present invention can be irradiated with light with a wave length adapted to generation of the photodynamic effect of the dye.

In Photodiagnosis Photodyn. Ther. (2018), 23, pp 347-352, Souza et al used photodynamic therapy to show antimicrobial activity of hypochlorite solutions and reciprocating instrumentation associated with photodynamic therapy on root canals infected with enterococcus faecalis. However, the test solutions was devoid of an antibacterial dye. These technologies are included in the present invention by reference.

Stepwise Method for Reduction of the Invention to Practice

The present invention provides compositions and methods of the use of solid precursors API-P of chlorinated species, combined with the activator, e.g. acetic acid or its salt, and methods of its use. Reduction of the invention to practice comprises the following 6 steps:
1. A pre-calculated amount of API-P having the general formula denoted below M [Cl(O)ₓ]ₙⁿ⁻, wherein M can be any alkali metal, alkaline earth metal or transition metal ion, wherein n is an integer 1-5, x is an integer 1-4, y is an integer 1-2. The solid state (API-P) generates a concentration of the API in the final solution in the form of an OC in the interval 0.01-1000 ppm, preferably in the range 0.1-100 ppm, is loaded into compartment 1 of a multi-compartment device. The API-P is mixed with a precalculated amount of NaCl to generate a final osmolality in the interval 0.1-500 mOsm, and optionally any other stabilizing solid.
2. A precalculated amount of an activator with the general formula R₁XOₙ(R₂)ₘ, wherein the activator is preferably acetic acid, optionally in a mixture with its metal or ammonium salt. The activator is dissolved in a pharmaceutically acceptable diluent, adjuvant, or carrier to generate a concentration of the activator in the interval 0.05-10%, preferably in the range 0.08 to 0.5%, even more preferably in the range 0.10-0.2%. If the API-P is not premixed with NaCl, the solution in step 2 may comprise an amount of NaCl from step, either way generating a final osmolality in the interval 0.1-500 mOsm, preferably around 300 mOsm, corresponding to 150 mM NaCl. An aliquot of the solution is loaded into a second compartment of a multi-compartment device.
3. To generate the main product according to the invention, compartment 1 and 2 are mixed by opening a port or breaking a seal, membrane barrier or between the first and second compartments to mix the contents in the compartments, followed by ambient squeezing or shaking to generate the disinfectant solution. The resulting solutions can be taken out through a cap on the multi-compartment device prior to use. The solution is isotonic, has a pH in the interval 4 to 9, preferably between 5 and 6, and is generally used for antimicrobial purposes, e.g. for inhalation therapy using e.g. an asthma inhaler or nebulizer to fight viral infections in the upper airways in mammals.
4. For applications where a color indicator in step 4. can add information in the therapeutic procedure, e.g. in treatment of mastitis, or for indication of the oxidative activity of the API, a dye with a color that varies with the oxidation state of the API (ROD), in a precalculated amount to generate a concentration of the dye in the concentration range 0.01-1000 ppm, is optionally loaded into an optional compartment of a multi-compartment device.
5. Depending on the intended use, a precalculated amount an amino acid as a stabilizer of the API, preferably taurine in the same concentration as the API, is optionally is optionally loaded into an optional compartment of a multi-compartment device. Step 4 is performed to reduce oxidative stress to biological surfaces.
6. Depending on the intended use, an amount of a water-soluble viscosity enhancer (VE) that cannot be oxidized by the API in the concentration range 0.01-25%, preferably in the range 0.1-10%, even more preferably in the range 0.2-1%, is mixed with the solution resulting from a selected sequence of steps 1-3, optionally combined with any of the steps step 4-5. A VE concentration of 0.01-0.1% generates a viscous but fluid solution, while 0.3-1% produce a gel. For skin or wound applications, the third compartment in step 3. comprising the VE can be included in the mixing procedure to produce a viscous or gel-formed product.

Use of the Invention in Agriculture

In agriculture, especially in animal farms, many kinds of infectious diseases caused by bacteria, viruses and fungi affect the daily operation of the farm, and affects the costs in running the facilities. In these settings, designed formulations according to the invention act therapeutically or prophylactically, and are especially useful in skin infections.

One important example is mastitis in cattle, which costs the US dairy industry about 1.7-2 billion USD each year. Effective and environmentally friendly treatment of mastitis has proven difficult, since milk from cows, having received long-term antibiotics is not marketable until the residual drugs have left the system. No vaccines are effective, since the infection in the udder and teats of the cow is remote from the animal's main blood stream. To mark cows having received treatment, dairy workers apply strips of tape to alert and mark treated cattle.

Thus, a preferred aspect of the present invention is treatment of mastitis using a gel or viscous solution comprising an OC, acetic acid or its salt, the viscosity enhancer VE and a ROD exemplified by methylene blue. The colored gel stays on the area of the udder and teats, acetic acid has the ability to penetrate into the skin of the teats, and the color makes use of strips of tape unnecessary. Additionally, the applied gel can be irradiated using light with suitable wavelength to increase the therapeutic effect of the gel. In this case, steps 1-4 and step 6 is performed to yield the instant formulation of use.

Use of the Invention in Aquaculture

Water quality is a prerequisite for a successful culture of aquatic animals, exemplified by fish, oysters, prawns and shrimps. Open water systems often bring organisms like virus, bacteria, lice, protozoa, fungal pathogens, algae and parasites. Common virus infections that lead to high mortality in aquatic species attractive for food production are Koi Herpes Virus Disease, Pancreas disease (PD) and infectious salmon anemia (ISA). Proper water quality or sufficient quantity of pure water is most often not available. The breeding installations in prior art often has no means of hindering these infectious species to approach and effect the breeding species. Further, once infected, there is no efficient cure to provide efficient therapy against these diseases.

A preferred embodiment of the oxidized chlorine species OC according to the present invention is effective treatment of all these infections and harmful organisms and cells. The instant formulations of the OC are highly effective in controlling these waterborne pathogens. In example, chlorine dioxide is a broad-spectrum biocide effective to solve the defined problems in the prior art. The formulations in the present invention is even employed in special tanks to repeatedly treat e.g. bred salmon without harming the fish gills or any other parts of the bred species, while having a destructive effect on the microorganisms causing the disease. In these applications, the preparations sequence wherein the API-P is NaOClO₂ or Ca(OClO₂)₂ is loaded into compartment 1 and mixed with a precalculated amount of acetic acid in step 1-3 is used.

Antiviral use of the invention The methods disclosed herein also permit improved methods of exposure of contaminated surfaces, equipment, e.g. medical equipment, any furniture surfaces, doorknobs, devices, clothing or personnel to disinfectant formulations employing misting or vaporization of the API into confined spaces. This is possible because the degradation status and quality of the disinfectant formulation is known, since it has not been stored as a solution, but is freshly prepared at site of interest from solid precursors.

This procedure ensures dispersion of the active agents into crevices and microenvironments, even onto personnel who are suspected of having been contaminated by infectious tissues or bodily fluids. Vaporization of these formulations may enable beneficial therapeutic or prophylactic impacts on resistant viral, bacterial or fungal infections.

Kim et al in Laryngoscope (2008), 118, pp 1862-1867, studied effects of a low concentration hypochlorous acid for nasal irrigation solution on bacteria, fungi, and virus. The formulations were also used in vitro against human rhinovirus (HRV) in nasal epithelial cells with significant virus-killing effect. Similar formulations were used with good effects against avian influenza virus through in vitro experiment as described by Hakim et al in J. Vet. Med. Sci. (2015), 77, pp 211-215.

Hypochlorous acid has also been used clinically in upper airway mucosa in rhinitis patients. Cho et al reported improved Outcomes after low-concentration hypochlorous acid after nasal irrigation in pediatric chronic sinusitis in Laryngoscope (2016), 126:791-795. To the best of our knowledge, similar formulations have not been used in prior art to treat other parts of respiratory system in mammals.

In U.S. Pat. No. 10,342,825 B by Robert Northey, a low pH antimicrobial solution comprising from 5 mg/L to 200 mg/L hypochlorous acid and water, wherein the solution has a pH of 5.6, stabilized by a phosphate buffer. The solutions may be vaporized using nebulizers for distribution on surfaces and tissue. However, a weakness the formulations used in prior art is the same as described above.

In WO 2019/222768 by Terry, A method for inactivating an infectious agent that is a resistant virus, a cancer-causing virus, a chemically resistant non-enveloped virus, or an infectious agent present in a mucous membrane or epithelial surface. The formulation comprises an infectious agent with a bufferless, electrolyzed, hypohalous acid composition. However, also in this case, a weakness the formulations used herein is the same as defined above.

Many of the problems in prior art have now been solved using the present invention. For the first time, OC has now been combined with an activator from solid precursors and NaCl for maintaining biological osmolality for the antiviral use of the resulting formulations.

The exposures can be affected without concerns for toxicity or corrosiveness which accompany prior methods of inactivation of highly contagious and resistant infectious agent types. A preferred embodiment is the eradicating, minimizing, or preventing progression of a viral infection in the upper airways, with the result that the immune system has time to mount an antibody response to the virus.

Thus, systems and methods of the invention provide oxidized chlorine, OC, as a means of treating viral infection in the respiratory tract. Compositions of the invention are capable of treating SARS, MERS and other infections, including but not limited to, SARS CoV-2 infections. This has now for the first time been facilitated through the instant precursors of the API combined with the multi-compartment device according to the invention, since there is no need to evaluate the lack of activity of a solution that has been stored at ambient conditions.

Particularly, inhalable hypochlorous acid formulations of OC, an activator, e.g. acetic acid, an excipient regulating the rheology of the final solution, an osmolality-regulating agent, e.g. sodium chloride. Such instant formulations can now be prepared on site, along with methods of delivery via a nebulizer, such as soft mist inhalers, jet nebulizers, ultrasonic wave nebulizers, and vibrating mesh nebulizers may be used. Upon use, inhalers and nebulizers aerosolize compositions of the invention for delivery via inhalation.

Formulations for aerosolization may be provided in dry powder form, solution, or suspension form. Fine droplets, sprays, and aerosols can be delivered by an intranasal or intrapulmonary pump dispenser or squeeze bottle. Compositions can also be inhaled via an inhaler, such as a metered dose inhaler or a dry powder inhaler. Compositions can also be inhaled via a nebulizer, such an ultrasonic wave nebulizer, providing compositions of OC and acetic acid directly to respiratory tracts via inhalable formulations. This prevents and treats infections of the respiratory system caused by viruses as well as other microbes. According to the invention, formulations as described herein are safe and effective for the prevention and treatment of viral infections.

Compositions of the invention may also include a pharmaceutically acceptable carrier, such as a diluent, to facilitate delivery to the respiratory mucosa. The carrier might be an aqueous carrier such as saline. The composition may be isotonic, having the same osmotic pressure as blood and lacrimal fluid. Suitable non-toxic pharmaceutically acceptable carriers are known to those skilled in the art. Various carriers may be particularly suited to different formulations of the composition, for example whether it is to be used as drops or as a spray, a suspension, or another form for pulmonary delivery.

Formulations for inhalation may be provided in dry powder form, solution, or suspension form. The composition can be delivered by various devices known in the art for administering drops, droplets, and sprays. The composition can be delivered by a dropper, pipet, or dispenser. Fine droplets, sprays, and aerosols can be delivered by an intranasal or intrapulmonary pump dispenser or squeeze bottle.

Intranasal delivery may be provided via a nasal spray device. Accordingly, the formulations according to the invention may be designed as a nasal spray. The nasal spray is insufflated into the nose and is delivered to the respiratory tract.

Soft mist inhalers use mechanical energy stored in a spring by user-actuation to pressurize a liquid container, causing the contained-liquid to spray out of a nozzle for inhalation in the form of a soft mist. Soft mist inhalers do not rely on gas propellant or electrical power for operation. The average droplet size in soft mist inhalers is about 5.8 micrometers.

Jet nebulizers are the most commonly used and may be referred to as atomizers. Jet nebulizers use a compressed gas (e.g., air or oxygen) to aerosolize a liquid medicine when released there through at high velocity. The resulting aerosolized droplets of therapeutic solution or suspension are then inhaled by a user for treatment. The compressed gas may be pre-compressed in a storage container or may be compressed on-demand by a compressor in the nebulizer.

Ultrasonic wave nebulizers rely on an electronic oscillator to generate a high frequency ultrasonic wave that, when directed through a reservoir of a therapeutic suspension of solution, aerosolized the medicine for inhalation.

Vibrating mesh nebulizers use the vibration of a membrane having thousands of holes at the top of the liquid reservoir to aerosolize a fine-droplet mist for inhalation. Vibrating mesh nebulizers avoid some of the drawbacks of ultrasonic wave nebulizers, offering more efficient aerosolization with reduced treatment times and less heating of the liquid being nebulized.

Treatment of a viral infection is achieved using a synergistic composition of acetic acid and hypochlorous acid. The acetic acid component is particularly effective for penetrating into tissues, while the hypochlorous acid is particularly effective for treating infection on the outer surface of tissue. As described above, these compositions are effective for treating the respiratory tract and for preventing respiratory infection.

The disclosed compositions are particularly effective because balancing the concentrations of hypochlorous acid and acetic acid with NaCl allows safe treatment of viruses. The precise balance depends on the formulation, the treatment site, and even the desired amount of surface penetration. The hypochlorous acid can be present in about 5 ppm up to about 1000 ppm or more. Different uses, different delivery methods, and types of tissue may require higher or lower concentrations. The acetic acid may be present at about 0.1-% up to about 5.0% or more, and preferably about 1.0%. By balancing the two components, the composition can have the dual effect of treating at the surface and beneath the surface of the tissue to which it is applied.

In the case that the OC is hypochlorous acid HOCl, an instant composition having a concentration of about 15-60 ppm of the OC is normally sufficient for treatment of infected lungs. In the case that the OC is chlorine dioxide OCl₂, a concentration of 0.1-5 ppm is usually sufficient.

In some cases, to fully destroy the virus or to prevent the virus from entering the respiratory tract, the composition should be in contact with it for a prolonged period, ranging from a few seconds, to several minutes, to an hour or more. Accordingly, in certain embodiments, the composition is in the form of a gel, which allows longer contact times with the infection site.

The use of the composition in combination with a known antiviral treatment may increase the efficacy of the compositions. In some embodiments, methods of the invention further comprise administration (simultaneously or sequentially with compositions of the invention) of one or more doses of an antiviral substance. These may include, but are not limited to, acyclovir, adefovir, adamantine, boceprevir, brivudin, cidofovir, emtricitabine, entecavir, famciclovir, fomivirsen, foscarnet, ganciclovir, lamivudine, penciclovir, telaprevir, telbivudine, tenofovir, valacyclovir, valganciclovir, vidarabine, m₂ inhibitors, neuraminidase inhibitors, interferons, ribavirin, nucleoside reverse transcriptase inhibitors, non-nucleoside reverse transcriptase inhibitors, non-structural protein 5a (ns5a) inhibitors, chemokine receptor antagonist, integrase strand transfer inhibitors, protease inhibitors, and purine nucleosides.

The compositions are also useful in combination with a known antimicrobial treatment. In some embodiments, methods of the invention further comprise administration (simultaneously or sequentially with compositions of the invention) of one or more doses of an antibiotic substance, including, but not limited to, ciprofloxacin, beta-lactam antibiotics like ampicillin or carbapenems, azithromycin, cephalosporin, doxycycline, fusidic acid, gentamycin, linezolid, levofloxacin, norfloxacin, ofloxacin, rifampin, tetracycline, tobramycin, vancomycin, amikacin, deftazidime, cefepime, trimethoprim/sulfamethoxazole, piperacillin/tazobactam, aztreanam, meropenem, colistin, or chloramphenicol.

In some embodiments, methods of the invention further comprise administration of one or more doses of an antibiotic substance from an antibiotic class including, but not limited to, aminoglycosides, carbacephem, carbapenems, first generation cephalosporins, second generatin cephalosporins, third generation cephalosporins, fourth generation cephalosporins, glycopeptides, macrolides, monobactam, penicillins, polypeptides, quinolones, sulfonamides, tetracyclines, lincosamides, and oxazolidinones. In some embodiments, methods of the invention comprise administration of a nonantibiotic antimicrobial substance, including but not limited to sertraline, racemic and stereoisomeric forms of thioridazine, benzoyl peroxide, taurolidine, and hexitidine.

The dosing regimen of the composition may include the amount, frequency, and duration of exposure to the composition. The dosing regimen may depend on the severity of the infection, or on a regimen prescribed for treatment or for prevention of the viral infection.

The composition may be administered in a single daily dose or in multiple doses, e.g., 2, 3, 4, or more doses, per day. The subject receiving the composition may be exposed to the composition for periods of hours or of minutes. The duration of exposure may depend on the frequency, amount, or even of the severity of the infection.

The total daily amount of API formed in the instant solution from the solid precursors may be in the range 0.01-1000 mg, depending of the nature of the OC. The actual dosage may vary depending upon the specific composition administered, the mode of administration, and other factors known in the art.

The composition may be administered to any member of the respiratory tract, such as the respiratory epithelium, nasal cavity, nasal epithelium, pharynx, esophagus, larynx, epiglottis, trachea, carina, bronchi, bronchioles, or the lungs. Administering the composition to the respiratory tract treats prevents any disease or disorder that is transmitted by a virus.

In certain other embodiments, the compositions of the invention can be used to disinfect whole rooms, facilities medical devices and surgical instruments, for example. Supplies of medical devices are often initially sterile, but may require additional or subsequent cleaning and disinfection or sterilization. In particular, sterilization or disinfection of reusable medical devices prior to reuse employing any known technique is especially important. Compositions can be applied to the medical device using. For example, the composition can be applied by wiping or spreading it onto the surface of the device, by spraying an aerosol or mist form of the composition onto the device, by dipping the device into a vessel containing a volume of the composition, or by placing the device into a flow of the composition such as from a faucet. Additionally or alternatively, medical devices and surgical instruments may also be stored submerged in the composition and removed at the time of use.

In summary, the disinfection efficacy of the complete instant formulation according to the invention have been found to be greater than the efficacy of its components alone, as known in prior art. The differences in performance are easily observable across a wide range of concentrations.

Additionally, since both oxidized chlorine chemical species and acetic acid is toxic at high concentrations, the prior art has taught away from the use of these agents on skin or other tissue, except in trace amounts. Thus, the present invention is surprisingly safe and effective when used in a controlled manner as described above.

Some of the disclosed compositions contain acetic acid at 2% or greater, and when in combination with the OC have proven to be safe and effective for treating skin and other tissues. The OC in these compositions has been found to have a modulating effect of the acetic acid. This allows the compositions to take advantage of the disinfecting properties of acetic acid without causing harm to the tissue.

### Incorporation by Reference

Any and all references and citations to other documents, such as patents, patent applications, patent publications, journals, books, papers, web contents, that have been made throughout this disclosure are hereby incorporated herein by reference in their entirety for all purposes.

### Equivalents

The invention may be embodied in other specific forms without departing from the spirit or essential characteristics thereof. The foregoing embodiments are therefore to be considered in all respects illustrative rather than limiting on the invention described herei

Experimental Part

### EXAMPLE 1

### General procedure for preparation of dry, air free solid mixtures of API-P and NaCl for aliquot loading into compartment 1 of the multi-compartment device.

1a. Production of Dry Powder Comprising 50 ppm Sodium Hypochlorite in Sodium Chloride

In 8.95 g of dry NaCl (mw: 58.44 g/mol), 50 mg of dry sodium hypochlorite (mw: 74.44 g/mol) was blended to a homogenous mixed powder and stored under air free and dry conditions in containers shielded from light. An aliquot of 90 mg of the powder is loaded into compartment 1 of the multi-compartment device.

1b. Production of Dry Powder Comprising 100 ppm Sodium Hypochlorite in Sodium Chloride

In 8.90 g of dry NaCl (mw: 58.44 g/mol), 100 mg of dry sodium hypochlorite (mw: 74.44 g/mol) was blended to a homogenous mixed powder and stored under air free and dry conditions in containers shielded from light. An aliquot of 90 mg of the powder is loaded into compartment 1 of the multi-compartment device.

1c. Production of Dry Powder Comprising 200 ppm Sodium Hypochlorite in Sodium Chloride

In 8.8 g of dry NaCl (mw: 58.44 g/mol), 200 mg of dry sodium hypochlorite (mw: 74.44 g/mol) was blended to a homogenous mixed powder and stored under air free and dry conditions in containers shielded from light. An aliquot of 90 mg of the powder is loaded into compartment 1 of the multi-compartment device.

1d. Production of Dry Powder Comprising 500 ppm Sodium Hypochlorite in Sodium Chloride

In 8.5 g of dry NaCl (mw: 58.44 g/mol), 500 mg of dry sodium hypochlorite (mw: 74.44 g/mol) was blended to a homogenous mixed powder and stored under air free and dry conditions in containers shielded from light. An aliquot of 90 mg of the powder is loaded into compartment 1 of the multi-compartment device.

1e. Production of Dry Powder Comprising 25 ppm Calcium Dihypochlorite in Sodium Chloride

In 8.975 g of dry NaCl (mw: 58.44 g/mol), 25 mg of dry calium hypochlorite (mw: 142.98 g/mol) was blended to a homogenous mixed powder and stored under air free and dry conditions in containers shielded from light. An aliquot of 90 mg of the powder is loaded into compartment 1 of the multi-compartment device.

1f. Production of Dry Powder Comprising 50 ppm Calcium Dihypochlorite in Sodium Chloride

In 8.975 g of dry NaCl (mw: 58.44 g/mol), 50 mg of dry calium hypochlorite (mw: 142.98 g/mol) was blended to a homogenous mixed powder and stored under air free and dry conditions in containers shielded from light. An aliquot of 90 mg of the powder is loaded into compartment 1 of the multi-compartment device.

1g. Production of Dry Powder Comprising 100 ppm Calcium Dihypochlorite in Sodium Chloride

In 8.9 g of dry NaCl (mw: 58.44 g/mol), 100 mg of dry calium hypochlorite (mw: 142.98 g/mol) was blended to a homogenous mixed powder and stored under air free and dry conditions in containers shielded from light. An aliquot of 90 mg of the powder is loaded into compartment 1 of the multi-compartment device.

1h. Production of Dry Powder Comprising 100 ppm Calcium Dihypochlorite in Sodium Chloride

In 8.9 g of dry NaCl (mw: 58.44 g/mol), 100 mg of dry calium hypochlorite (mw: 142.98 g/mol) was blended to a homogenous mixed powder and stored under air free and dry conditions in containers shielded from light. An aliquot of 90 mg of the powder is loaded into compartment 1 of the multi-compartment device.

1i. Production of Dry Powder Comprising 100 ppm Calcium Dihypochlorite in Sodium Chloride

In 8.9 g of dry NaCl (mw: 58.44 g/mol), 100 mg of dry caliutn hypochlorite mw: 142.98 g/mol) was blended to a homogenous mixed powder and stored under air free and dry conditions in containers shielded from light. An aliquot of 90 mg of the powder is loaded into compartment 1 of the multi-compartment device.

1j. Production of Dry Powder Comprising 1 ppm Sodium Chlorite in Sodium Chloride

In 89.99 g of dry NaCl (mw: 58.44 g/mol), 10 mg of dry sodium chlorite (mw: 90.44 g/mol) was blended to a homogenous mixed powder and stored under air free and dry conditions in containers shielded from light. An aliquot of 90 mg of the powder is loaded into compartment 1 of the multi-compartment device.

1k. Production of Dry Powder Comprising 5 ppm Sodium Chlorite in Sodium Chloride

In 89.99 g of dry NaCl (mw: 58.44 g/mol), 50 mg of dry sodium chlorite (mw: 90.44 g/mol) was blended to a homogenous mixed powder and stored under air free and dry conditions in containers shielded from light. An aliquot of 90 mg of the powder is loaded into compartment 1 of the multi-compartment device.

1l. Production of Dry Powder Comprising 10 ppm Calcium Chlorite in Sodium Chloride

In 89.99 g of dry NaCl (mw: 58.44 g/mol), 100 mg of dry calcium chlorite (mw: 157.89 g/mol) was blended to a homogenous mixed powder and stored under air free and dry conditions in containers shielded from light. An aliquot of 90 mg of the powder is loaded into compartment 1 of the multi-compartment device.

### EXAMPLE 2

### General procedure for preparation of 1 L stock solutions of activator for low-volume aliquot loading into compartment 4, 5 or 10 of the multiple compartment device

2a. Acetic Acid Solution (0.125%, pH 2.95)

In 998.75 mL of sterile water, 1.25 mL of acetic acid (mw: 60.05 g/mol) was dissolved.

2b. Acetic Acid Solution (0.125%, pH 4.3)

In 998.75 mL of sterile water, 1.25 mL of acetic acid (mw: 60.05 g/mol) was dissolved. The pH was adjusted to 4.3 using 10 N NaOH.

2c. Acetic Acid Solution (0.25%, pH 4.3)

998.75 mL of sterile water, 2.5 mL of acetic acid (mw: 60.05 g/mol) was dissolved. The pH was adjusted to 4.3 using 10 N NaOH.

2d. Acetic Acid Solution (0.25%, pH 5.0)

In 998.75 mL of sterile water, 2.5 mL of acetic acid (mw: 60.05 g/mol) was dissolved. The pH was adjusted to 5.0 using 10 N NaOH.

2e. Acetic Acid Solution (1%, pH 4.3)

998.75 mL of sterile water, 10 mL of acetic acid (mw: 60.05 g/mol) was dissolved. The pH was adjusted to 4.3 using 10 N NaOH.

2f. Acetic Acid Solution (2%, pH 4.3)

In 998.75 mL of sterile water, 20 mL of acetic acid (mw: 60.05 g/mol) was dissolved. The pH was adjusted to 4.3 using 10 N NaOH.

2g. Acetic Acid/Sodium Acetate Solution (0.1 M, pH 5.0)

In 800 mL of distilled water, 5.772 g of sodium acetate (mW: 82 g/mol), 1.778 g of acetic acid (mw: 60.05 g/mol) was added to the solution. The pH was adjusted to 5.0 using 10N HCl or 10 N NaOH, and distilled water was added until the volume was 1 L.

2h. Isotonic Acetic Acid Solution (0.125%, pH 2.95)

In 998.75 mL of sterile water, 1.25 mL of acetic acid (mw: 60.05 g/mol) and 8.4 g NaCl (mw: 58.44 g/mol) was added.

2i. Isotonic Acetic Acid Solution (0.125%, pH 4.3)

In 998.75 mL of sterile water, 1.25 mL of acetic acid (mw: 60.05 g/mol) and 8.4 g NaCl (mw: 58.44 g/mol) was added. The pH was adjusted to 4.3 using 10 N NaOH.

2j. Isotonic Acetic Acid Solution (0.25%, pH 4.3)

In 998.75 mL of sterile water, 2.5 mL of acetic acid (mw: 60.05 g/mol) and 8.4 g NaCl (mw: 58.44 g/mol) was added. The pH was adjusted to 4.3 using 10 N NaOH.

2k. Isotonic Acetic Acid Solution (0.125%, pH 5.0)

In 998.75 mL of sterile water, 1.25 mL of acetic acid (mw: 60.05 g/mol) and 8.4 g NaCl (mw: 58.44 g/mol) was added. The pH was adjusted to 5.0 using 10 N NaOH.

2l. Isotonic Acetic Acid Solution (0.25%, pH 5.0)

In 998.75 mL of sterile water, 2.5 mL of acetic acid (mw: 60.05 g/mol) and 8.4 g NaCl (mw: 58.44 g/mol) was added. The pH was adjusted to 5.0 using 10 N NaOH

2m. Isotonic Acetic Acid/Sodium Acetate Solution (0.1 M, pH 5.0)

In 800 mL of distilled water, 5.772 g of sodium acetate (mW: 82 g/mol), 1.778 g of acetic acid (mw: 60.05 g/mol) and and 8.4 g NaCl (mw: 58.44 g/mol) was added to the solution. The pH was adjusted to 5.0 using 10N HCl or 10 N NaOH, and distilled water was added until the volume was 1 L.

2n. Acetate Buffer (0.1 M, pH 5.0)

In 800 mL of sterile water, 5.772 g of sodium acetate (mW: 82 g/mol) and 1.778 g of acetic acid (mw: 60.05 g/mol) was added to the solution. The pH was adjusted to 5.0 using 10N HCl, and distilled water was added until the volume was 1 L.

2o. ACES Buffer (0.1 M, pH 6.7)

In 800 mL of sterile water, 18.22 g of N-(2-acetamido)-2-aminoethanesulfonic acid (mW: 182.2 g/mol) was added to the solution. The pH was adjusted to 6.7 using pH using 10 N NaOH, and distilled water was added until the volume was 1 L.

2p. Citric Acid Solution (0.1 M, pH 2.2)

An amount of 19.2 g of citric acid (mw: 192.1 g/mol) was dissolved In 2L of sterile water.

2q. Citrate Buffer (0.1 M, pH 6.0)

In 800 mL of sterile water, 12.044 g of sodium citrate (mW: 294.1 g/mol) and and 11.341 g of citric acid (mw: 192.1 g/mol) was added to the solution. The pH was adjusted to 6.0 using 0.1 N NaOH, and distilled water was added until the volume was 1 L.

2r. ADA Buffer (0.1 M, pH 6.6)

In 800 mL of sterile water, 95.11 g of 2-[(2-amino-2-oxoethyl)-(carboxymethyl)amino]acetic acid (ADA, mW: 190.22 g/mol) was added to the solution. ADA dissolved when the pH was adjusted to 6.6 using pH using 10N NaOH, and distilled water was added until the volume was 1 L.

2s. EBBS Buffer Including the Dye Phenol Red (pH 7.0)

In 800 mL of sterile water, 200 mg of CaCl₂ (mW: 110.98 g/mol), 200 mg of MgSO₄-7H₂O (mW: 246.47 g/mol), 400 mg of KCl (mW: 75 g/mol), 2.2 g of NaHCO₃ (mW: 84.01 g/mol), 6.8 g of NaCl (mw: 58.44 g/mol), 140 mg NaH₂PO₄H₂O (mw: 138 g/mol), 1 g D-Glucose (Dextrose) (mw: 180.16 g/mol) and 10 mg phenol red Phenol Red (mw: 354.38 g/mol) was added to the solution. The pH of the solution was adjusted to 7.0 or another desired pH using HCl or NaOH.

2t. Sterile Isotonic Oxygenated Water

A stock volume of 1 L of sterile water saturated with oxygen is added 9 g of NaCl and stored at room temperature in a sealed bottle shielded from light.

### EXAMPLE 3

### Instant Preparation of Ready to Use Disinfectant Formulations from Solid Salts of Oxidized Chlorine Combined with Solutions from Example 1

### EXAMPLE 3.1

### Non-Limiting Steps of a General Procedure

1. An aliquot of 90 mg of any of the powders from example 1 is loaded into compartment 4 of the multi-compartment device.

2. An aliquot of 10 mL of any of the activator solutions from Example 2 is loaded into compartment 4 of the multi-compartment device.

3. To generate the main product according to the invention, the seal, barrier or port 3 according to FIG. 1 between the screw cap and compartment 4 is broken or opened to mix the contents in compartment 1 with the solution in compartment 4, followed by gently squeezing or shaking to generate the disinfectant solution. The resulting solution can be taken out through the opening after removing the screw cap on the multi-compartment device, and are now ready to use. The isotonic solutions have a pH in the interval 4 to 9, preferably between 5 and 6, is generally used for antimicrobial purposes.

4. Optionally, depending on the intended use, a water-soluble dye in solid form with a color that varies with the oxidation state of the API (ROD), in a precalculated amount to generate a concentration of the dye in the concentration range 0.01-1000 ppm, is optionally loaded into compartment 9 of the multi-compartment device, and the procedure in 3. is repeated including mixture of compartments 1,4 and 9.

5. Optionally, depending on the intended use, a precalculated amount an amino acid as a stabilizer of the API, preferably taurine in the same concentration as the API, is optionally loaded into compartment 5 of the multi-compartment device. and the procedure in 3. is repeated including mixture of compartments 1,4 and 5.

6. Optionally, depending on the intended use, e.g. for skin or wound applications, an amount of a water-soluble viscosity enhancer (VE) that cannot be oxidized by the API, precalculated to gain a concentration of VE in the final solution in the concentration range 0.01-25%, is loaded into compartment 5 of the multi-compartment device. A VE concentration of 0.01-0.1% generates a viscous but fluid solution, while 0.3-1% produce a gel. The dispersion of the VE in the solution from step 3, 4 and/or 5 is converted to a viscous solution or a gel using a Silverson Mixer or an Ystral Mixer, and used on site for skin or wound applications. The viscous solution or a gel have increased stability because of slower motions of molecules and may be packed into soft bags, bottles protecting the solution or gel from air and light for later use.

### EXAMPLE 4

### In Vitro Anti-Biofilm Effect of Example Three Different Test Solutions of HOCl and Acetic Acid

Three different test solutions were generated form the multi-compartment device. All three test solutions are generated as described in example 3.1 from the multicompartment device, loaded with 90 mg of dry powder comprising 200 ppm sodium hypochlorite in sodium chloride (example 1c) in compartment 1. Three aliquots of 10 mL of acetic acid solutions from (0.125%, pH 4.3, example 2b) in compartment 4 in three different multicompartment-devices. Solution 1: (0.25%, pH 4.3, example 2c), Solution 2: (1%, pH 4.3, example 2e), Solution 3: (2%, pH 4.3, example 20.

Experimental Setup

Test organisms: Pseudomonas aeruginosa or Staphylococcus aureus wild-type strains Biofilm type: 48 hours- or 24 hours-old biofilms grown on semipermeable membranes placed on solidified medium supplemented with 0.5% glucose. In the case of 48 h-old biofilms, the membranes with biofilms were transferred onto fresh plates after 24 h.

Initial viable cell amount: 5×10⁹ colony forming units (CFUs)

Treatment method: Membranes with biofilms were transferred to new plates. Eight-10 layers of sterile gauze were placed on the second membrane, and 1 ml of antimicrobial solution was pipetted on the gauze layers. The treatment was carried out at room temperature for 2-to-3 h, or 4-to-6 h. In the case of the 4-to-6 h treatments, the gauze layers were replaced with fresh gauze layers with lml sample solution 2 or 3 h after the treatments had been initiated.

Evaluation method: The gauze layers were discarded, and each membrane with biofilms was transferred into a 15 ml tube containing 5 ml 0.9% NaCl, vortexed for 10 sec., sonicated in an ultrasound bath for 10 min, and vortexed again for 10 sec. Ten-fold serial dilutions were made, and 10 ul of each dilution was spot-plated on LB plates for viable CFU counting.

Results and Conclusions

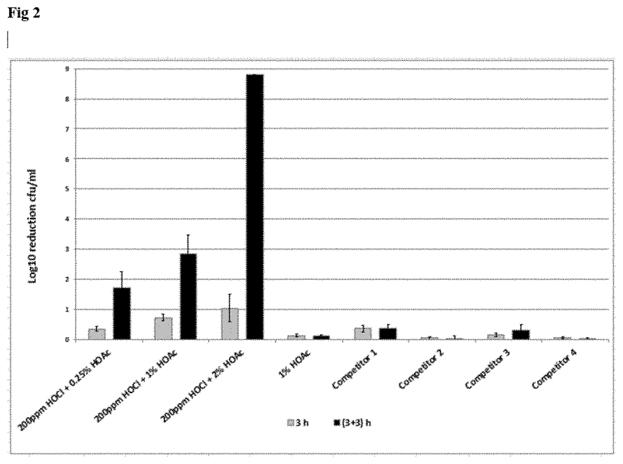

FIG. 2 shows the results obtained using the sample solutions. Increasing the HAc concentrations from 0.25% to 1% and 2% in a 200 ppm HOCl solution gradually increased the killing of S. aureus biofilms. The effect of 1% acetic acid alone had only minor effect on the biofilm. The three test solutions were compared to 4 different competing wound healing products on the market which all showed only minor effects on the S. aureus biofilms. An even stronger effect was shown for biofilms from P. Aeruginosa It is concluded that hypochlorous acid and acetic acid at pH 4.3 acts synergistically and efficiently at concentrations that have shown to be safe in other studies.

### EXAMPLE 5

### In Vitro Antiviral Effect of Example 1+2.

### EXAMPLE 6

### In Vitro Antifungal Effect of Example 1+2.

### EXAMPLE 7

### In Vivo Toxicity Studies

### EXAMPLE 7.1

### 7 Day Inhalation Toxicity Study in Rats

A 7 day inhalation toxicity study in rats is performed as described by Kogel et al in 2913 in https://www.pmiscience.com/resources/docs/default-source/default-document-library/2013_ukogel_ict_poster.pdf?sfvrsn=d6a9f606_0 The rat inhalation study is performed according to the Organization for Economic Cooperation and Development (OECD). The test solution is generated as described in example 3.1 from the multicompartment device, loaded with 90 mg of dry powder comprising 100 ppm sodium hypochlorite in sodium chloride (example 1b) in compartment 1 and an aliquot of 10 mL of acetic acid solution (0.125%, pH 4.3, example 2b) in compartment 4. Test Guideline 412, Sprague-Dawley rats is exposed to filtered fresh air (sham) as a reference, or the test solution. Care and use of the animals is in accordance with the American Association for Laboratory Animal Science Policy (1996). All animal experiments are approved by the Institutional Animal Care and Use Committee (IACUC). The histopathological evaluation is performed at defined anatomical sites of the nose and of the left lung according to a defined grading system. Free lung cells are determined in bronchoalveolar lavage fluid by flow cytometry, and inflammatory mediators are measured by multi-analytes profiling (MAP). For the Systems Toxicology approach, RNA samples from specific sites in the respiratory tract are obtained, i.e., respiratory nasal epithelium (RNE) and lung. For lung RNA isolation, respiratory epithelium of main bronchus and lung parenchyma is separated by Laser Capture Microdissection (LCM) and further processed, and analyzed on whole genome Affymetrix microarrays (GeneChip® Rat Genome 230 2.0 Array).No major perturbations are found related to inflammation, cell stress, cell proliferation in bronchi or lung parenchyma.

### EXAMPLE 8

### Treatment of Mastitis

For applications where a color indicator in step 4. can add information in the therapeutic procedure, e.g. in or for indication of the oxidative activity of the API, the compartment comprising the ROD is included in the procedure.

### EXAMPLE 9

### Clinical Antiviral Therapy

The medicine cup of Gima Aerosol Corsia Nebulizer is loaded with 5 mL of the test solution generated as described in example 3.1 from the multicompartment device, loaded with 90 mg of dry powder comprising 1 ppm sodium chlorite in sodium chloride (example 1j) in compartment 1 and an aliquot of 10 mL of citric acid solution (0.1 M, pH 2.2, example 2p) in compartment 4. The mouth of a patient with a corona virus lung infection is attached to the hose and the face mask attached to the nebulizer, which is started After 10-15 minutes of breathing, the fluid is used up, and the nebulizer is turned off. The patient is monitored for several hours to secure that no side effects of the treatment is taking place. The mucosa and cilia of the patient is investigated for potential side effects.

1. A disinfectant composition comprising:
a solid oxidized chlorine species salt according to the formula:
Mⁿ⁺[Cl(O)ₓ]ₙⁿ⁻
where M can be any alkali metal, alkaline earth metal or transition metal ion, n is an integer 1-2, x is an integer 1-4;
an activator with the general formula:
R₁XOₙ(R₂)ₘ
where the group R₁ may be a group comprising 1-10 hydrogenated carbon atoms, optionally substituted with amino, amido, carboxylic, sulfonic or hydroxy groups, where group X may be a carbon, phosphorous or sulfur atom, n and m may be an integer 2 or 3 and R₂ may be a proton H, or any alkali metal, alkaline earth metal or transition metal ion salt or ammonium salt; and
a pharmaceutically-acceptable diluent, adjuvant, or carrier.

2. The composition of claim 1, wherein said oxidized chlorine salt comprises an alkali metal or alkaline earth metal salt of hypochlorous acid HOCl.

3. The composition of claim 2, wherein said activator is acetic acid.

4. The composition of claim 1, wherein said oxidized chlorine salt comprises an alkali metal or alkaline earth metal salt of chlorous acid HOClO.

5. The composition of claim 4, wherein said activator is acetic acid.

6. The composition of claim 1, wherein said activator is acetic acid.

7. The composition of claim 1, wherein the composition comprises an osmolality in the range of about 0.1 mOsm to about 500 mOsm.

8. The composition of claim 1, wherein an amount of oxidized chlorine species salt, acetic acid or its metal or ammonium salt produces a pH between 4 and 8.

9. The composition of claim 1, further comprising a viscosity-enhancing agent.

10. The composition of claim 9, wherein the viscosity-enhancing agent cannot be oxidized by the oxidized chlorine species.

11. The composition of claim 9, wherein the viscosity-enhancing agent comprises a water- soluble gelling agent.

12. The composition of claim 9, wherein the water-soluble gelling agent is selected from the group consisting of poly acrylic acid, polyethylene glycol, poly(acrylic acid)-acrylamidoalkylpropane sulfonic acid co-polymer, phosphino polycarboxylic acid, and poly(acrylic acid)-acrylamidoalkylpropane or sulfonic acid-sulfonated styrene terpolymers.

13. The composition of claim 1, further comprising a dye including a colour providing a visual indication of the presence an oxidized chlorine compound.

14. The composition of claim 11, wherein the dye comprises a reduction-oxidation dye.

15. The composition of claim 11, wherein colour and intensity of colour of the dye is dependent on an oxidation state of the oxidized chlorine compound.

16. The composition of claim 1, wherein the composition is formulated in an aqueous solution, gel, cream, ointment, or oil.

17. The composition of claim 1, wherein the composition is produced and stored in a multi- compartment container.

18. The composition of claim 1, wherein fluid and solid components are contained within separate respective compartments prior to combination of said fluid and solid components to produce said composition.

19. The composition of claim 1, wherein use of the formulation is for at least one of: antimicrobial applications; inhalation therapy using an asthma inhaler, nebulizer or vaporizer to fight viral infections in the upper airways in mammals; skin or wound prophylaxis or healing; treatment of mastitis or any other infectious disease in animal or agricultural breeding; and antiviral applications.

20. The composition of claim 1, wherein the composition is formulated in an aqueous solution, gel, cream, ointment, or oil.
